(19) United States
(12) Patent Application Publication
(10) Pub. No.: US 2019/0341150 A1
(43) Pub. Date: Nov. 7, 2019

(54) Automated Radiographic Diagnosis Using a Mobile Device

(71) Applicant: Google LLC, Mountain View, CA (US)

(72) Inventor: Hormuz Mostofi, San Francisco, CA (US)

(21) Appl. No.: 15/968,282

(22) Filed: May 1, 2018

(51) Int. Cl. G16H 50/20; G16H 70/60; G06N 3/08; G06N 3/04; G06T 7/00; G06K 9/62; G06K 9/00

(52) U.S. Cl. CPC G16H 50/20; G16H 70/60; G06N 3/08; G16H 50/70; G06T 7/0014; G06K 9/6215; G06K 9/00671; G06N 3/04

(57) ABSTRACT

A wireless device, an app on a wireless device, and a method for automated diagnosis of radiographs is described. The app prompts a user to capture a photograph of a radiograph external to the mobile device with the mobile device's camera. The quality of the photograph is assessed and an error condition is reported if the quality is insufficient. A module displays on the mobile device display (1) a diagnosis that is assigned to the radiographs and (2) at least one similar radiograph. The diagnosis is assigned by subjecting the photograph to a deep learning model trained on a large corpus of labelled radiographs. The deep learning model can be resident on the mobile device or in a back end server. The app includes tools for enabling the user to select and navigate the input photograph and the similar radiograph by means of hand gestures on the display, and a tool for displaying medical knowledge associated with the diagnosis.

This disclosure relates to a method of generating a diagnosis for a radiograph (either digital or analog) using machine learning and providing the diagnosis to a mobile computing device such as a smartphone. The features of this disclosure supports non-radiologists, such as X-ray technicians, or nurses, who are often tasked to interpret radiographs due to shortages of trained radiologists, particularly in developing countries, or radiologists or even lay persons that may lack specialized training. The method can be implemented in an app residing on a mobile device, or in a combination of a mobile device and a back-end server.

Remote radiographic diagnosis using JPEG-formatted radiographs transmitted to smartphones is known, see Peter G. Noel et al., OFF-SITE SMARTPHONE VS. STANDARD WORKSTATION IN THE RADIOGRAPHIC DIAGNOSIS OF SMALL INTESTINAL MECHANICAL OBSTRUCTION IN DOGS AND CATS, Vet Radiol Ultrasound, Vol. 57, No. 5, 2016, pp 457-461. Other prior art includes A. Rodriguez et al. Radiology smartphone applications; current provision and cautions, Insights Imaging (2013) 4:555-562; and G. Litgens et al., A Survey on Deep Learning in Medical Image Analysis arXiv:1702.05747v2 [cs.CV] 4 Jun. 2017.

The term “mobile device” is intended to be interpreted to cover portable electronic communication devices, such as smartphones, personal digital assistants, tablet computers and wifi only devices. Typically, such devices will include at least a camera, a touch sensitive display, wireless communication technology to connect to computer networks (e.g., 4G, LTE, wifi and the like currently), and a central processing unit which executes apps loaded on the device. The following discussion will describe the mobile device as a smartphone, but it will be understood that the methods can be used with other types of mobile devices, e.g., a tablet computer.

### SUMMARY

In one aspect of this disclosure, a mobile device is described which includes a camera, a processing unit, a touch-sensitive display, and a memory storing instructions for an app executed by the processing unit. The app includes:

a) a prompt for the user to capture at least one photograph of one or more analog or digital radiographs external to the mobile device with the camera;

b) an image quality assessment module for assessing the quality of the at least one photograph captured by the camera and reporting an error condition if the quality of the at least one photograph is insufficient;

c) a module for displaying on the display (1) a diagnosis assigned to the one or more analog or digital radiographs and (2) at least one similar radiograph associated with the diagnosis, wherein the diagnosis is assigned by subjecting the at least one photograph to a deep learning model trained on a large corpus of radiographs;

d) tools for enabling the user to select the at least one similar radiograph associated with the diagnosis and navigate within at least one similar radiograph by means of hand gestures on the display; and

e) a tool for displaying medical knowledge associated with the diagnosis on the display.

In another aspect, a method for providing diagnostic information for radiographic images on a mobile device having a camera and a display is described. The method includes the steps of:

(a) assessing the image quality of at least one photograph of one or more analog or digital radiographic images taken by the camera;

(b) reporting an error condition if the quality of the at least one photograph is insufficient;

(c) subjecting the at least one photograph to a deep learning model trained on a large corpus of radiographs and generating a diagnosis for the at least one photograph;

(d) identifying at least one radiograph image similar to the at least one photograph having the diagnosis;

(e) displaying on the display (1) the diagnosis generated by the deep learning model in step (c) and (2) the at least one similar radiograph image identified in step (d);

(f) providing tools on the mobile device enabling the user to select the at least one similar radiograph image associated with the diagnosis and navigate within the at least one similar radiograph image by means of hand gestures on the display; and

(g) providing a tool for displaying medical knowledge associated with the diagnosis on the display.

In still another aspect, there is disclosed an app for a mobile device having a camera, a processing unit, a touch-sensitive display, and a memory storing instructions for an app executed by the processing unit, wherein in the app comprises:

a) a prompt presented on the display for the user to capture at least one photograph of one or more analog or digital radiographs external to the mobile device with the camera; and

b) an image quality assessment module for assessing the quality or suitability of the at least one photograph captured by the camera for processing by a deep learning diagnostic model, the assessment module reporting an error condition if the quality or suitability of the at least one photographs is insufficient.

### BRIEF DESCRIPTION OF THE DRAWINGS

FIG. 1 is an overview illustration of the use of a mobile device, in this instance a smartphone, for radiographic diagnosis in accordance with one embodiment in which deep learning models on a remote computer network are used to return a diagnosis and similar medical images. In an alternative embodiment the deep learning models and similar medical images are stored locally on the smartphone.

FIG. 2 is a block diagram of a smartphone having the features of this disclosure.

FIG. 3 is a block diagram of a radiology app loaded on the smartphone of FIG. 2.

FIG. 4 is a flow chart showing one embodiment of the method of this disclosure.

FIG. 5 is an illustration of a home display of a radiology app on a smartphone, where the user is provided with options to select different types of radiographs for diagnosis.

FIG. 6 is an illustration of a display of the radiology app showing a prompt to capture a photograph of an external chest X-ray, either digital or analog, with the smartphone camera. Tools on the display allow the user to select capturing either a single image or multiple images.

FIG. 7 is an error message produced by an image quality assessment module optionally implemented in the smartphone indicating that the image captured in FIG. 6 is of insufficient quality to perform the machine learning algorithms and generate a diagnosis.

FIG. 8 is a display of a possible diagnoses generated by a deep learning model for the image captured as per FIG. 6, along with similar medical images (chest X-rays) obtained from other patients for each of the possible diagnoses.

FIG. 9 is a “compared input” display on the app showing the input image captured by the smartphone camera side by side with one of the similar images shown in FIG. 8.

FIG. 10 is a display of additional medical knowledge on the smartphone relating to the diagnosis returned in FIG. 8; the additional medical knowledge is accessed by activating the LEARN MORE icon of FIG. 2, 8 or 9.

### DETAILED DESCRIPTION

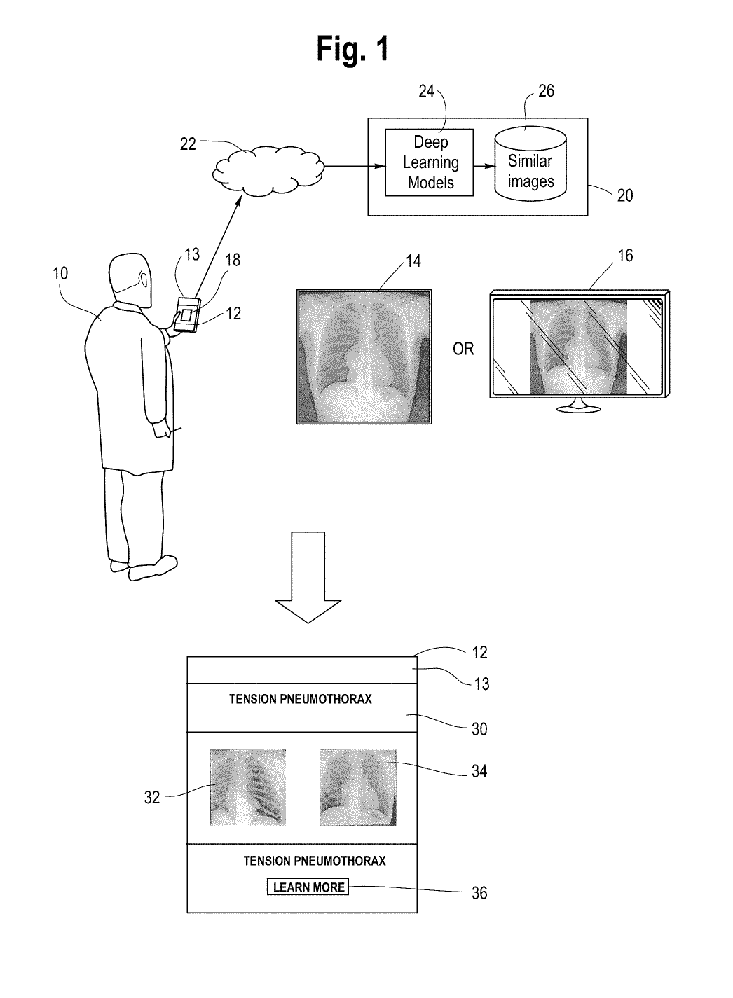

An overview of the method of automatic radiographic diagnosis using a mobile device will be now described in conjunction with FIG. 1. In the method, a user 10 (typically a non-radiologist, such as a technician, nurse or general practitioner, or even a patient), has a mobile device (e.g., smartphone, tablet computer, etc.) 12 equipped with a camera (not shown in FIG. 1) and a touch sensitive display 13, as is conventional. An app, described below, is resident on the smartphone which provides the prompts, displays, and optionally machine learning aspects of the method described herein. A prompt is displayed on the display asking the user to take a photograph of either an analog X-ray film 14, or a digital X-ray presented on a computer screen 16, with the smartphone camera. In some cases, images are captured of multiple X-rays, such as anterior/posterior and lateral X-ray views, e.g., in a chest X-ray diagnostic scenario. The input image can be captured with the camera in still camera mode, or in a video camera mode in which case a frame of imagery is grabbed by the app, for example when the user is using the camera in a camera mode and viewing the camera image on the display.

The quality of the image(s) 18 captured by the smartphone camera is assessed, e.g., with a convolutional neural network. This assessment is optionally done locally on the smartphone by execution of an image quality assessment module which is part of the app. If the image is of poor quality or unfeasible for interpretation or processing by machine learning algorithms, an error is immediately returned and displayed on the smartphone to the user. Various reasons for rejection of an image include user error (improper window/level settings of a radiograph on a digital display, too much glare on the digital display, camera focus issues) or radiography technique related quality problems (inspiration issues, patent rotated, inclusion issues, radiograph over or under exposed, etc.). The error message can include prompts or instructions for how to overcome the error condition.

Once acceptable (high quality) photographs of radiographic images are captured by the smartphone camera and input in the app, they are supplied to a deep convolutional neural network that has been trained on a large corpus of X-ray images labeled with ground truth annotations of diagnosis. The deep convolutional network infers a diagnosis (or potential alternative diagnoses). The inferred diagnosis, or alternative diagnoses, is returned to the smartphone along with similar X-ray images from other patients grouped by diagnosis. In one configuration, the smartphone is connected with a back end server 20 via a cellular data network and connected networks (such as the Internet). In this configuration, a service provider implements the back end server 20 which contains the deep convolutional neural network 24 to generate the diagnosis and a data store 26 which contains a multitude of ground truth labelled radiographic images, one or more of which are selected by the neural network for transmission to the smartphone 12. The back end server 20 could also implement the image quality assessment module in one possible configuration.

In one possible configuration, the smartphone 12 is configured with a local, lightweight deep convolutional neural network model such as MobileNet such that the deep learning model 24 can run locally without adversely sacrificing precision and recall. See e.g. Andrew G. Howard et al., MobileNets: Efficient Convolutional Neural Networks for Mobile Vision Applications, arXiv:1704.04861 [cs.CV] (Apr. 17, 2017). Running the deep learning convolutional neural network locally on the mobile device has the advantage that no cell service or back end server connection is required to generate the diagnosis or display the related images, for example in situations where the method is implemented in remote areas.

The app on the smartphone contains includes a module for displaying the diagnosis 30 (in this example, “tension pneumothorax”), the input image 32 (in this example, a chest X-ray) and one or more similar images 34 in various formats. In one format, the similar images are grouped by diagnostic findings in order of acuity. A tool such as an arrow 37 or scroll bar allows the user to scroll through and see the related similar images returned along with the diagnosis. The user can also tap on any of the similar images 34 or tap a tool such as “compare input” and proceed to an image comparison screen where the input image captured on the smartphone is shown adjacent to the similar image(s).

Additional tools are provided on the app for viewing the similar image or the input image:

a) the user can pan/zoom around by means of single finger up/down gestures on the images.

b) the user can window or level with two finger up/down or left/right gestures.

c) the user can proceed to view the next similar image by tapping on the arrows on the left and right of the displayed similar image. The similar images can be panned and zoomed to display the relevant region of interest with similar findings/diagnosis.

The app further includes additional medical learning information tools by which they can obtain more information about the findings/diagnoses proposed for the input image. For example, the app can display a LEARN MORE icon 36 next to the input image 32 and if the icon 36 is selected the app displays an explanation of the underlying pathology with recommended management.

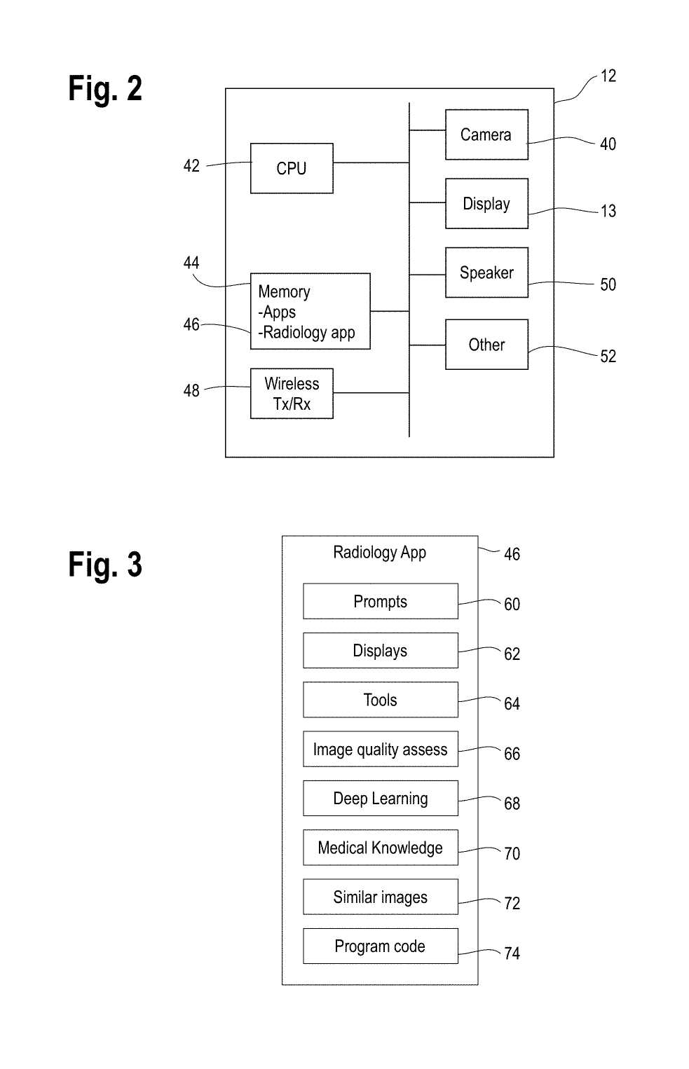

FIG. 2 is a block diagram of one example of a mobile device 12 configured to perform the method described above. The mobile device is in the form of smartphone 12 which includes a camera 40, the display 13, a central processing unit 42, a memory 44 storing apps and program code including a radiology app 46 used in the method of FIG. 1, wireless transmit and receive circuits 48 (conventional), a speaker 50 and other conventional circuits 52, the details of which are not important.

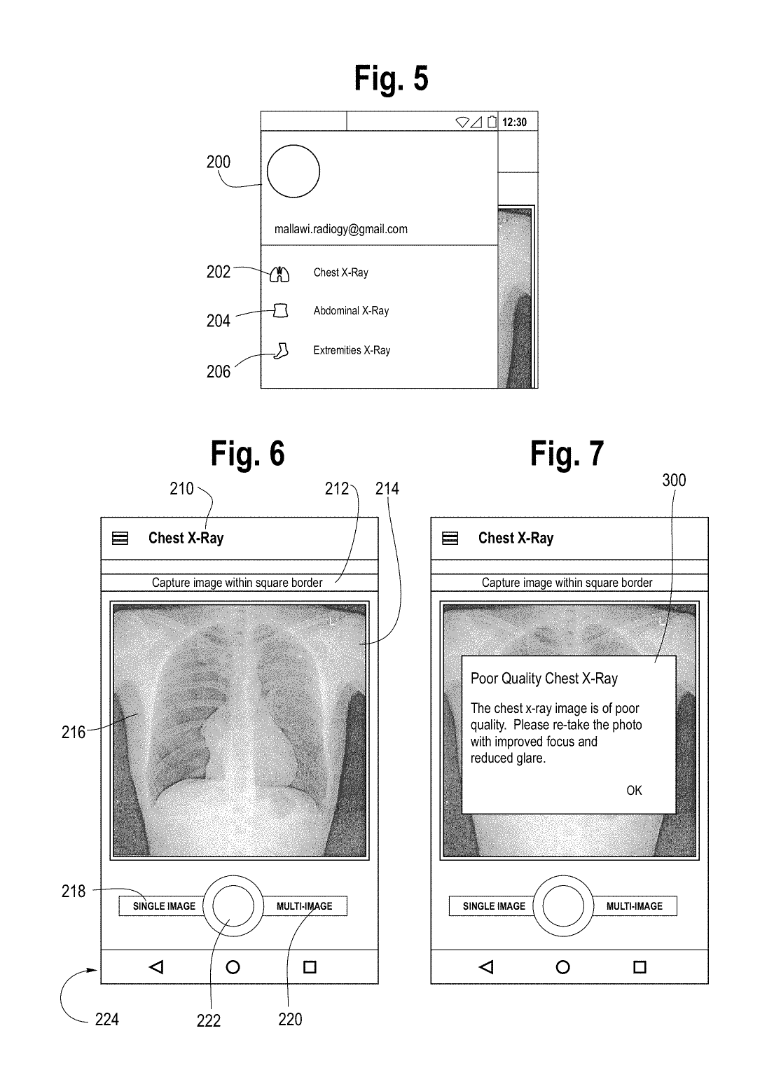

FIG. 3 is a block diagram of the radiology app 46 loaded on the smartphone 12 of FIG. 2. The app includes prompts 60 for prompting the user to take certain action, such as capture a photograph a radiograph with the smartphone's camera. The app includes displays 62 for displaying the prompts, the input image captured by the camera, and other features as will be explained with the screen shots of FIGS. 5-10. The app further includes tools 62, including hand gesture tools which work in conjunction with the touch sensitive display for enabling the user to navigate through the input image or similar images, or to take other action as explained in conjunction with FIGS. 5-10. The app further includes an image quality assessment module 66, implemented as a convolutional neural network, which assesses the quality of radiographic images captured by the smartphone camera for suitability for use by machine learning algorithms to generate a diagnosis. The network is trained to detect error conditions, such as user errors for example not capturing sufficient amount of the radiograph, insufficient illumination, too much glare, camera focus issues, or radiography technique quality problems (e.g., inspiration issues, patent rotated, inclusion issues, radiograph over or under exposed, etc.). The image quality assessment module can have the same general architecture of the deep convolutional neural network trained to generate the list of diagnoses as described at some length below or in the cited scientific and patent literature. If no such error conditions are noted by the image quality assessment module, the input image is supplied to a deep learning module 68, for example a lightweight deep convolutional neural network (e.g., MobileNet) trained on a large corpus of radiographic images with ground truth labels in order to generate a diagnosis for the input radiographic image.

The app 46 further includes a store of medical knowledge 70 in the form of text or text plus images which are pertinent to the diagnoses the deep learning module 68 is trained to make. The medical knowledge can consist of descriptions of the diagnoses, along with treatment or response guidelines, as well as alert prompts to prompt the user to take certain action in the case that the diagnosis is such that the patient associated with the input radiographic image requires immediate medical attention.

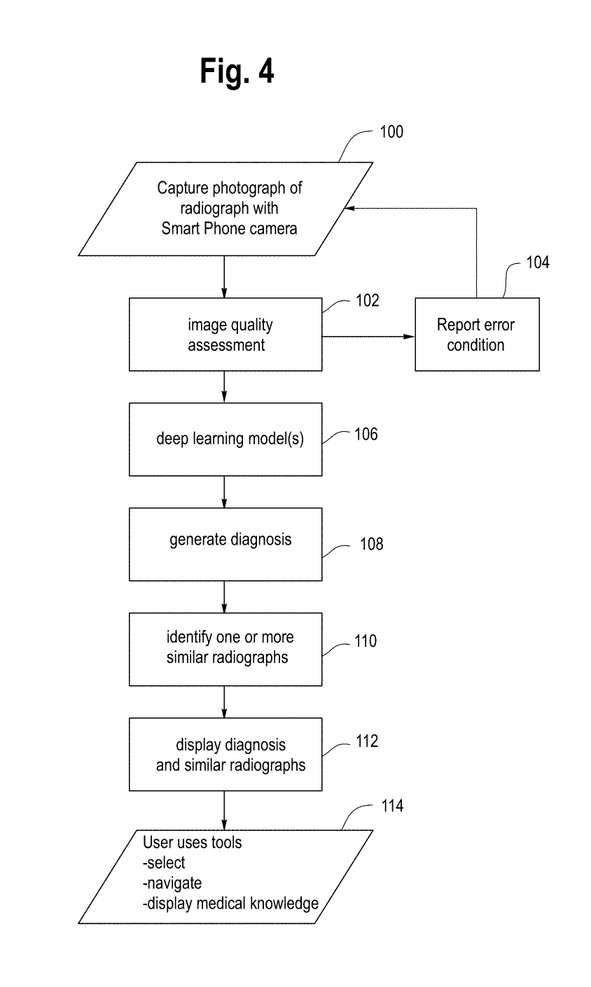

The app 46 further includes a data store 72 of similar medical images. For example, in a chest X-ray scenario, the data store may include hundreds of stored radiographic images of patients with various diagnoses from chest X-rays. Each of the images could be associated with metadata for the images, such as the diagnosis, treatment information, age of the patient, smoker status, etc. The app further includes program code 74 to present the displays in the sequence indicated by the logic of FIG. 4 and the descriptions of FIGS. 5-10 below. Such code can be developed by persons skilled in the art given the present disclosure.

FIG. 4 is a flow chart showing one embodiment of the method of this disclosure. At step 100, the user is prompted to capture a photograph of a radiograph, which could be in either analog form or digital form and e.g. displayed on a computer monitor. The user captures the photograph with the smartphone camera. At step 102, an image quality assessment module 66 (FIG. 3) is invoked which determines whether error conditions are present in the image. If so, at step 104 the smartphone reports an error condition and prompts the user to correct the error, e.g. by reducing glare, providing greater illumination, etc. and the user is again prompted to take an image at step 100.

If no error condition is detected at step 102, the input image is passed to a deep learning model 106. For example, the deep learning model could be resident in a back end server as shown in FIG. 1 or the deep learning model could be implemented in a lightweight format on the smartphone, see FIG. 3 at 68. The model is trained to perform two tasks: 1) generate a diagnosis, step 108 and 2) identify similar radiographic images 110 in the data store to the input image and having the same diagnosis. In one alternative, there could be two different deep learning models, one to perform step 108 and one to perform step 110.

Machine learning models for searching for similar medical images are described in the literature, see for example: J. Wang, et al., Learning fine-grained image similarity with deep ranking, https://arxiv.org/abs/1404.4661 (2017), and the literature cited therein; PCT application PCT/US18/25054 filed Mar. 29, 2018, and the following US patent documents: U.S. Pat. Nos. 9,275,456; 9,081,822; 7,188,103; 2010/0017389; 2007/0258630; 2003/0013951.

Machine learning models for generating a diagnosis from radiographic images is described in PCT application PCT/US2018/018509 filed Feb. 16, 2018. Such machine learning models (FIG. 1, 24; FIG. 3; 68, FIG. 4; 106) can be implemented in several different configurations. One implementation is the Inception-v3 deep convolutional neural network architecture, which is described in the scientific literature. See the following references, the content of which is incorporated by reference herein: C. Szegedy et al., Going Deeper with Convolutions, arXiv:1409.4842 [cs.CV] (September 2014); C. Szegedy et al., Rethinking the Inception Architecture for Computer Vision, arXiv:1512.00567 [cs.CV] (December 2015); see also U.S. patent application of C. Szegedy et al., “Processing Images Using Deep Neural Networks”, Ser. No. 14/839,452 filed Aug. 28, 2015. A fourth generation, known as Inception-v4 is considered an alternative architecture. See C. Szegedy et al., Inception-v4, Inception-ResNet and the Impact of Residual Connections on Learning, arXiv:1602.0761 [cs.CV] (February 2016). See also U.S. patent application of C. Vanhoucke, “Image Classification Neural Networks”, Ser. No. 15/395,530 filed Dec. 30, 2016. The description of the convolutional neural networks in these papers and patent applications is incorporated by reference herein. Another alternative for the deep learning model is described in the paper of X. Wang et al., ChestX-ray8: Hospital-scale Chest X-ray Database and Benchmarks on Weakly-Supervised Classification and Localization of Common Thorax Diseases, arXiv:1705.02315v5 [cs.CV] (December 2017), the content of which is incorporated by reference.

Referring again to FIG. 4, at step 112 the diagnosis for the input image and the similar images are displayed on the display of the smartphone 12. At step 114, the user uses tools on the smartphone, e.g., to select similar images to study further, to navigate around the input image or the similar images, or to display stored medical knowledge relating to the diagnosis, e.g., in order to learn more about the diagnosis or the management or treatment of the condition reflected in the diagnosis.

The screen shots of FIGS. 5-10 will now be described which show one particular configuration of the app residing on the mobile device. It will be appreciated that the screen shots are offered by way of illustration of one possible manner in which the methods of this disclosure can be practiced, and are in no way limiting.

FIG. 5 is an illustration of a home display 200 of a radiology app (app 46, FIG. 2) on a mobile device 12. The user is provided with options to select different types of radiographs for diagnosis, for example by activating the icon 202 the user is indicating they will be capturing chest X-rays, the icon 204 indicates the user will be captured abdominal X-rays, and by activating the prompt 206 the user indicates they will be capturing X-rays of extremities. By activating one of the icons, a particular deep learning model trained for that type of X-ray is flagged such that the input images are directed to the pertinent deep learning model.

Assume now the user has selected the chest X-ray icon 202. At this point, the app presents the display 210 of FIG. 6 on the smartphone. The user is prompted via the prompt 212 to capture a photograph of an external analog or digital chest X-ray with their smartphone camera within the square border region 214. The chest X-ray image 216 represents the viewfinder image that the camera captures. The user can activate the SINGLE IMAGE icon 218 to indicate they will be capturing a single image. The user can activate the MULTI IMAGE icon 220 to indicate they will capture more than one image. By pressing on the central circle area 222 the smartphone camera is operated to capture the photograph. Navigation tools 224 on the bottom of the display allow the user to navigate back to the home screen or go back to a previous screen.

After the image 216 is captured it is processed by the image quality assessment module 66 (FIG. 3) preferably locally on the smartphone. FIG. 7 depicts an error message 300, produced by an image quality assessment module optionally implemented in the smartphone, indicating that the image captured in FIG. 6 is of insufficient quality to perform the machine learning algorithms and generate a diagnosis. In this example, the image was captured out of focus and will too much glare. The error message includes instructions on how to overcome the error condition, e.g. with the text “PLEASE RETAKE THE PHOTO WITH IMPROVED FOCUS AND REDUCED GLARE.”

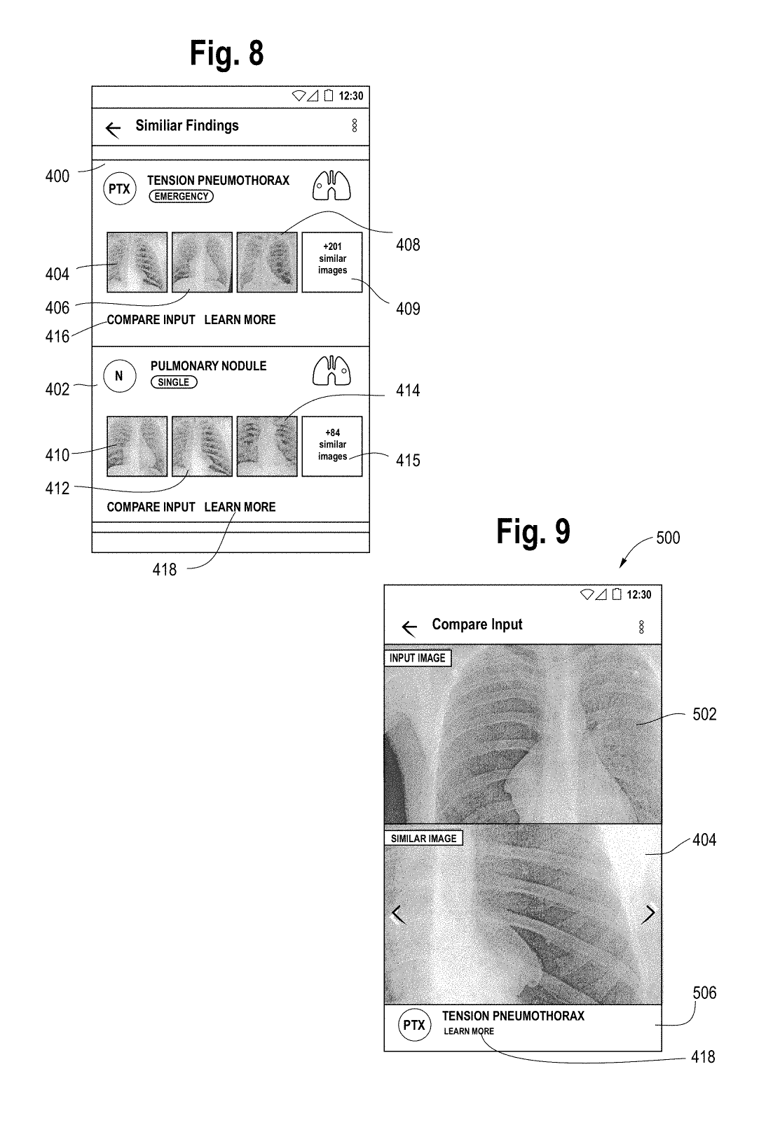

FIG. 8 is a display of possible diagnoses 400, 402 generated by a deep learning model (68, FIG. 2) for the image captured as per FIG. 6, along with similar medical images (chest X-rays) 404, 406, 410, 412 obtained from other patients for each of the possible diagnoses. In this example, the first listed diagnosis 400 is tension pneumothorax, and that diagnosis is displayed along with three similar images below that (404, 406 and 408) along with an icon 409 stating that over two hundred additional similar images are available for viewing. Below that there is listed the diagnosis pulmonary nodule, along with similar images 410, 412, 414 and an icon 415 stating that eighty four similar images are available for viewing for that diagnosis. By using hand gestures on the display the user can scroll down to see other possible diagnosis and associated similar images.

Each diagnosis has a COMPARE INPUT icon 416 which allows for side by side comparison of the input image and one of the similar images, see FIG. 9. Also, each diagnosis has a LEARN MORE icon 418 which when activated generates a display of medical knowledge associated with the diagnosis.

FIG. 9 is a “compared input” display 500 on the app showing the input image 502 captured by the smartphone camera side by side (in this case, above) one of the similar images shown in FIG. 8, in this case image 404 from FIG. 8. The user can use hand gestures to navigate around the display of the input image 502 or the similar image, e.g., pan, zoom in, zoom out, using one or two figure gestures. The diagnosis is displayed in the lower region 506. A LEARN MORE icon 418 is displayed which when activated changes the display to a display of text or text plus graphics/images of medical knowledge relating to the diagnosis.

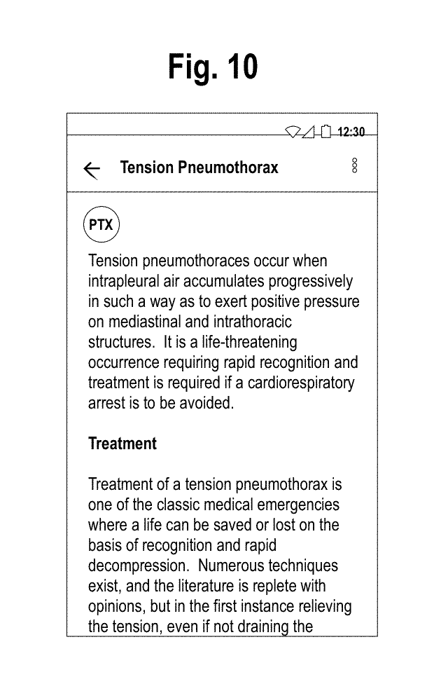

FIG. 10 is a display of additional medical knowledge on the smartphone relating to the diagnosis returned in FIG. 8; the additional medical knowledge is accessed by activating the LEARN MORE icon 418 of FIG. 8 or 9.

Further considerations:

In one possible implementation, if a diagnosis is returned that indicates the patient is associated with the radiograph is in need of urgent medical attention the diagnosis screen (FIG. 8) can include alerts or prompts the have the patient seek immediate medical attention. In one configuration, the alert can include a link to real-time, on-line medical support service provider or medical hotline to call. This is shown in FIG. 8 with the EMERGENCY notification below the diagnosis TENSION PNEUMOTHORAX.

In view of the above, it will be apparent that a mobile device 12 has been described comprising: a camera 40 (FIG. 2), a processing unit 42 (FIG. 2), a touch-sensitive display 13 (FIGS. 1, 2, 5-10); and a memory 46 (FIG. 2) storing instructions for an app 46 (FIG. 2, 3) executed by processing unit 42, wherein in the app includes:

a) a prompt (FIG. 6, 212) for the user to capture at least one photograph of one or more analog or digital radiographs external to the mobile device with the camera;

b) an image quality assessment module (FIG. 3, 66) for assessing the quality of the at least one photograph captured by the camera and reporting an error condition if the quality of the at least one photographs is insufficient;

c) a module for displaying on the display (1) a diagnosis assigned to the one or more analog or digital radiographs and (2) at least one similar radiograph associated with the diagnosis, wherein the diagnosis is assigned by subjecting the at least one photograph to a deep learning model trained on a large corpus of radiographs, (FIG. 8, display module 62 FIG. 3);

d) tools for enabling the user to select the at least one similar radiograph associated with the diagnosis and navigate within at least one similar radiograph by means of hand gestures on the display (e.g., by exercising hand gestures to touch the images of FIG. 8 or use the COMPARE INPUT icon 416 of FIG. 8); and

e) a tool for displaying medical knowledge associated with the diagnosis on the display (e.g. the LEARN MORE icon 418 of FIG. 8, FIG. 10).

It will be apparent that an app for a mobile device (app 46, FIGS. 2, 3, 5-10) having features a)-e) above has also been described.

There has also been described a method for providing diagnostic information for radiographic images on a mobile device having a camera and a display, comprising the steps of:

(a) assessing the image quality of at least one photograph of one or more analog or digital radiographic images taken by the camera (FIG. 4 step 102);

(b) reporting an error condition if the quality of the at least one photograph is insufficient (FIG. 4 step 104);

(c) subjecting the at least one photograph to a deep learning model trained on a large corpus of radiographs and generating a diagnosis for the at least one photograph (FIG. 4, step 106, 108);

(d) identifying at least one radiograph image similar to the at least one photograph having the diagnosis (FIG. 4 step 110);

(e) displaying on the display (1) the diagnosis generated by the deep learning model in step (c) and (2) the at least one similar radiograph image identified in step (d) (FIG. 4 step 112, FIG. 8);

(f) providing tools on the mobile device enabling the user to select the at least one similar radiograph image associated with the diagnosis and navigate within the at least one similar radiograph image by means of hand gestures on the display (see description of FIG. 8, including icons and displayed images which can be touched and manipulated); and

(g) providing a tool for displaying medical knowledge associated with the diagnosis on the display (LEARN MORE icon of FIG. 8, 9 and knowledge presented in FIG. 10)

The ability of a mobile device to determine locally the suitability of captured images to be used by trained deep learning diagnostic models is independently useful and advantageous. Accordingly, in another aspect of this disclosure there is provided an app for a mobile device having a camera, a processing unit, a touch-sensitive display, and a memory storing instructions for an app executed by the processing unit, wherein in the app includes a) a prompt presented on the display for the user to capture at least one photograph of one or more analog or digital radiographs external to the mobile device with the camera (e.g., FIG. 6 icon 212); and b) an image quality assessment module 66 (FIG. 3) for assessing the quality or suitability of the at least one photograph captured by the camera for processing by a deep learning diagnostic model, the assessment module reporting an error condition if the quality or suitability of the at least one photographs is insufficient.

I claim:

1. A mobile device, comprising:
a camera;
a processing unit,
a touch-sensitive display; and
a memory storing instructions for an app executed by processing unit, wherein in the app comprises:
a) a prompt for the user to capture at least one photograph of one or more analog or digital radiographs external to the mobile device with the camera;
b) an image quality assessment module for assessing the quality of the at least one photograph captured by the camera and reporting an error condition if the quality of the at least one photographs is insufficient;
c) a module for displaying on the display (1) a diagnosis assigned to the one or more analog or digital radiographs and (2) at least one similar radiograph associated with the diagnosis, wherein the diagnosis is assigned by subjecting the at least one photograph to a deep learning model trained on a large corpus of radiographs;
d) tools for enabling the user to select the at least one similar radiograph associated with the diagnosis and navigate within at least one similar radiograph by means of hand gestures on the display; and
e) a tool for displaying medical knowledge associated with the diagnosis on the display.

2. The mobile device of claim 1, wherein the one or more analog or digital radiographs comprise a chest X-ray.

3. The mobile device of claim 1, wherein the one or more analog or digital radiographs comprise an abdominal X-ray.

4. The mobile device of claim 1, wherein the one or more analog or digital radiographs comprise an X-ray of a body extremity.

5. The mobile device of claim 1, wherein the deep learning model trained on a large corpus of radiographs is resident on the mobile device.

6. The mobile device of claim 1, wherein the deep learning model trained on a large corpus of radiographs is resident on a back end server.

7. The mobile device of claim 1, further comprising a store of a multitude of radiographic images, and wherein the at least one similar radiograph associated with the diagnosis is retrieved from the store.

8. The mobile device of claim 1, wherein the display displays the diagnosis and a plurality of similar radiographs from different patients grouped together with the display of the diagnosis.

9. The mobile device of claim 1, wherein the image quality assessment module is configured to detect both user errors in capturing the at least one photograph and errors in the at least one radiograph.

10. Apparatus comprising an app for a mobile device having a camera, a processing unit, a touch-sensitive display, and a memory storing instructions for an app executed by processing unit, wherein in the app comprises:
a) a prompt presented on the display for the user to capture at least one photograph of one or more analog or digital radiographs external to the mobile device with the camera;
b) an image quality assessment module for assessing the quality of the at least one photograph captured by the camera and reporting an error condition if the quality of the at least one photographs is insufficient;
c) a module for displaying on the display (1) a diagnosis assigned to the one or more analog or digital radiographs and (2) at least one similar radiograph associated with the diagnosis, wherein the diagnosis is assigned by subjecting the at least one photograph to a deep learning model trained on a large corpus of radiographs;
d) tools for enabling the user to select the at least one similar radiograph associated with the diagnosis and navigate within at least one similar radiograph by means of hand gestures on the display; and
e) a tool for displaying medical knowledge associated with the diagnosis on the display.

11. The app of claim 10, wherein the one or more analog or digital radiographs comprise a chest X-ray.

12. The app of claim 10, wherein the one or more analog or digital radiographs comprise an abdominal X-ray.

13. The app of claim 10, wherein the one or more analog or digital radiographs comprise an X-ray of a body extremity.

14. The app of claim 10, wherein the app further comprises the deep learning model trained on a large corpus of radiographs.

15. The app of claim 10, wherein the app further comprises a store of a multitude of radiographic images, and wherein the at least one similar radiograph associated with the diagnosis is retrieved from the store.

16. The app of claim 10, wherein the image quality assessment module is configured to detect both user errors in capturing the at least one photograph and errors in the at least one radiograph.

17. A method for providing diagnostic information for radiographic images on a mobile device having a camera and a display, comprising the steps of:
(a) assessing the image quality of at least one photograph of one or more analog or digital radiographic images taken by the camera;
(b) reporting an error condition if the quality of the at least one photograph is insufficient;
(c) subjecting the at least one photograph to a deep learning model trained on a large corpus of radiographs and generating a diagnosis for the at least one photograph;
(d) identifying at least one radiograph image similar to the at least one photograph having the diagnosis;
(e) displaying on the display (1) the diagnosis generated by the deep learning model in step (c) and (2) the at least one similar radiograph image identified in step (d);
(f) providing tools on the mobile device enabling the user to select the at least one similar radiograph image associated with the diagnosis and navigate within the at least one similar radiograph image by means of hand gestures on the display; and
(g) providing a tool for displaying medical knowledge associated with the diagnosis on the display.

18. Apparatus comprising an app for a mobile device having a camera, a processing unit, a touch-sensitive display, and a memory storing instructions for an app executed by the processing unit, wherein the app comprises:
a) a prompt presented on the display for the user to capture at least one photograph of one or more analog or digital radiographs external to the mobile device with the camera; and
b) an image quality assessment module for assessing the quality or suitability of the at least one photograph captured by the camera for processing by a deep learning diagnostic model, the assessment module reporting an error condition if the quality or suitability of the at least one photographs is insufficient.

19. The apparatus of claim 18, wherein the image quality assessment module is configured to detect both user errors in capturing the at least one photograph and errors in the at least one radiograph.

20. The apparatus of claim 18, wherein the deep learning diagnostic model is trained to diagnosis conditions in chest, abdominal cavity, or extremity X-rays.
